(19) United States
(12) Patent Application Publication
(10) Pub. No.: US 2013/0202657 A1
(43) Pub. Date: Aug. 8, 2013

(54) FOAMS OR PARTICLES FOR APPLICATIONS SUCH AS DRUG DELIVERY

(71) Applicants: Kosta Ladavac, Somerville, MA (US); Rodrigo E. Guerra, Cambridge, MA (US); David Kaz, Somerville, MA (US); Vinothan Manoharan, Cambridge, MA (US); Jens B. Rieger, Ludwigshafen (DE); Roland S. Koltzenburg, Dannstadt (DE); David A. Weitz, Bolton, MA (US)

(72) Inventors: Kosta Ladavac, Somerville, MA (US); Rodrigo E. Guerra, Cambridge, MA (US); David Kaz, Somerville, MA (US); Vinothan Manoharan, Cambridge, MA (US); Jens B. Rieger, Ludwigshafen (DE); Roland S. Koltzenburg, Dannstadt (DE); David A. Weitz, Bolton, MA (US)

(73) Assignees: BASF SE, Ludwigshafen (DE); President and Fellows of Harvard College, Cambridge, MA (US)

(21) Appl. No.: 13/697,692

(22) PCT Filed: May 20, 2011
(86) PCT No.: PCT/US11/37363

§ 371 Date: Jan. 28, 2013

Related U.S. Application Data
(60) Provisional application No. 61/347,062, filed on May 21, 2010.

(51) Int. Cl. A61K 9/12

(52) U.S. Cl. CPC A61K 9/122
USPC 424/400; 514/396; 514/254.07; 514/543; 514/217; 514/182

(57) ABSTRACT

The present invention generally relates to foams and, in particular, to foams for applications such as drug delivery, and particles that are made from such foams. One aspect relates to foams or particles containing pharmaceutically active agents. The foam may comprise a pharmaceutically acceptable polymeric carrier. In some cases, the foam or particle has an unexpectedly high specific surface area. A high specific surface area may, in some cases, facilitate delivery or release of the pharmaceutically active agent when the foam or particles made from the foam (e.g., by milling) are administered to a subject. The foam may also exhibit a relatively high loading of the pharmaceutically active agent. In some cases, the foam may be a microcellular foam. In one set of embodiments, the foam is created using a supercritical fluid, such as supercritical C02. For example, a precursor to the foam, containing a pharmaceutically active agent, may be mixed with a foaming agent, then the pressure decreased to cause the foaming agent to expand, thereby causing a foam to form. The foam may then be subsequently ground or milled, or otherwise processed to form particles.

### RELATED APPLICATIONS

This application claims the benefit of U.S. Provisional Patent Application Ser. No. 61/347,062, filed May 21, 2010, entitled “Foams or Particles For applications Such as Drug Delivery,” by Ladavac, et al., incorporated herein by reference.

### FIELD OF INVENTION

The present invention generally relates to foams and, in particular, to foams for applications such as drug delivery, and particles that are made from such foams.

### BACKGROUND

Nanoscale particles are of interest to applications such as drug delivery because of their high surface-to-volume ratio. But making nanoscale particles typically involves precipitation and growth. The problem with such methods is that the growth process is difficult to stop, and different precipitation processes are required for different ingredients. Accordingly, improvements in the creation of nanoscale particles are needed.

### SUMMARY OF THE INVENTION

The present invention generally relates to polymeric foams for applications such as drug delivery, and particles that are made from such foams. The subject matter of the present invention involves, in some cases, interrelated products, alternative solutions to a particular problem, and/or a plurality of different uses of one or more systems and/or articles.

In one aspect, the present invention is generally directed to a pharmaceutically active article. According to one set of embodiments, the pharmaceutically active article includes a foam comprising a pharmaceutically acceptable polymeric carrier and a pharmaceutically active agent. In some embodiments, the foam has an average cell size of less than about 5 micrometers and/or a specific surface area of at least about 0.4 m²/g. The pharmaceutically active agent, in some cases, may be present in the foam in an amount of at least about 5% based on the weight of the foam.

The pharmaceutically active article, in another set of embodiments, includes a plurality of particles. In some cases, the plurality of particles comprises a pharmaceutically acceptable polymeric carrier. In certain embodiments, the particles comprise a pharmaceutically active agent and/or the particles have an average characteristic dimension of no more than about 5 micrometers and/or a specific surface area of at least about 6 m²/g. In some embodiments, at least about 20% of the particles have at least one or at least two concave surface regions. In other embodiments, in at least about 20% of the particles, at least about 50% of the external surface area of the particles is present within a concave surface region.

In yet another set of embodiments, the pharmaceutically active article includes a foam comprising at least about 30 wt % of a pharmaceutically active agent. In some cases, the foam comprises a pharmaceutically acceptable polymeric carrier. In some embodiments, the foam has an average cell size of less than about 5 micrometers and/or the foam has a specific surface area of at least about 0.4 m²/g and/or the foam has a foam density of less than about 1 g/cm³.

The pharmaceutically active article, according to still another set of embodiments, includes a foam comprising a pharmaceutically acceptable polymeric carrier and a pharmaceutically active agent. In some embodiments, the foam has an average cell size of less than about 5 micrometers and/or the foam has a foam density of less than about 1 g/cm³. In certain cases, the pharmaceutically active agent is present in the foam in an amount of at least about 5 wt % based on the weight of the foam.

In one set of embodiments, the pharmaceutically active article comprises a foam comprising a pharmaceutically acceptable polymeric carrier and a pharmaceutically active agent, where the foam has an average cell size of less than about 5 micrometers. In some cases, the foam (a) has a specific surface area of at least about 0.4 m²/g, and/or (b) has a foam density of less than about 1 g/cm³.

In another set of embodiments, the pharmaceutically active article, comprises a plurality of particles, where the particles comprise a pharmaceutically acceptable polymeric carrier and a pharmaceutically active agent and have an average characteristic dimension of no more than about 5 micrometers and a specific surface area of at least about 6 m²/g. In some cases, (a) at least about 20% of the particles have at least two concave surface regions, and/or (b) in at least about 20% of the particles, at least about 50% of the external surface area of the particles is present within a concave surface region.

Another aspect of the present invention is generally directed to a method of forming a pharmaceutically active article. According to certain embodiments, the method includes acts of mixing a pharmaceutically acceptable polymeric carrier and a pharmaceutically active agent with a foaming agent to form a precursor of a foam, and subjecting the precursor to a pressure drop whereby the foaming agent expands and forms the pharmaceutically active article as a foam of the precursor. In one set of embodiments, the foam is microcellular. In some cases, the foaming agent is present in an amount of at least about 5% by weight based on the weight of the mixture. In certain embodiments, the pharmaceutically active agent is present in an amount of at least about 5% based on the weight of the mixture.

In another aspect, the present invention is directed to a method of making one or more of the embodiments described herein, for example, a polymeric foam such as a microcellular foam or other types of foams or particles as discussed herein. In another aspect, the present invention is directed to a method of using one or more of the embodiments described herein, for example, a polymeric foam such as a microcellular foam or other types of foams or particles as discussed herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B show various foam structures and particles in accordance with certain embodiments of the invention;

FIGS. 2A-2C illustrate various foam morphologies, according to certain embodiments;

FIG. 3 illustrates certain foams prepared in accordance with various embodiments of the invention;

FIG. 4 shows foams prepared in accordance with certain embodiments of the invention;

FIG. 5 illustrates ground preparations in accordance with still other embodiments of the invention;

FIGS. 6A-6B illustrate dissolution data in yet other embodiments of the invention;

FIGS. 7A-7B illustrate grain size distributions of certain foams, in yet another embodiment of the invention; and

FIGS. 8A-8B illustrate certain thin film foams, in yet another set of embodiments.

### DETAILED DESCRIPTION

The present invention generally relates to foams and, in particular, to foams for applications such as drug delivery, and particles that are made from such foams. One aspect relates to foams or particles containing pharmaceutically active agents. The foam may comprise a pharmaceutically acceptable polymeric carrier. In some cases, the foam or particle has an unexpectedly high specific surface area. A high specific surface area may, in some cases, facilitate delivery or release of the pharmaceutically active agent when the foam or particles made from the foam (e.g., by milling) are administered to a subject. The foam may also exhibit a relatively high loading of the pharmaceutically active agent. In some cases, the foam may be a microcellular foam. In one set of embodiments, the foam is created using a supercritical fluid, such as supercritical CO₂. For example, a precursor to the foam, containing a pharmaceutically active agent, may be mixed with a foaming agent, then the pressure decreased to cause the foaming agent to expand, thereby causing a foam to form. The foam may then be subsequently ground or milled, or otherwise processed to form particles.

In certain aspects, particles such as nanoparticles may be created and controlled by using foaming techniques to constrain particle formation. In one set of embodiments, foams are created, where the material between cells or bubbles within the foam is controlled. The size of the cells or bubbles and/or the packing density of these may be controlled to control the intercellular spacing within the resulting foam, thereby controlling the size or shape of the particles or nanoparticles that are created using the foam. For instance, although the cells or bubbles within a foam may be controlled to be on the micrometer scale, when the bubbles are closely packed together, the spaces between them (e.g., the “plateau regions”), where the material defining the foam is located, may be on the nanoscale. This material can include, for example, a polymer containing a pharmaceutically active agent (i.e., the “active”). In some embodiments, a high specific surface area may be achieved by controlling the size and/or packing density of the cells or bubbles in order to make very small domains of active-laden polymer within a foam. These cells or bubbles may be small (e.g., about 1 micron diameter) and highly packed (e.g., ˜85% volume fraction), yielding borders of few hundred nanometers, or polymeric foam films below about 50 nm thick. Such foams may then be processed to form particles, for example, by grinding or milling the foam, etc.

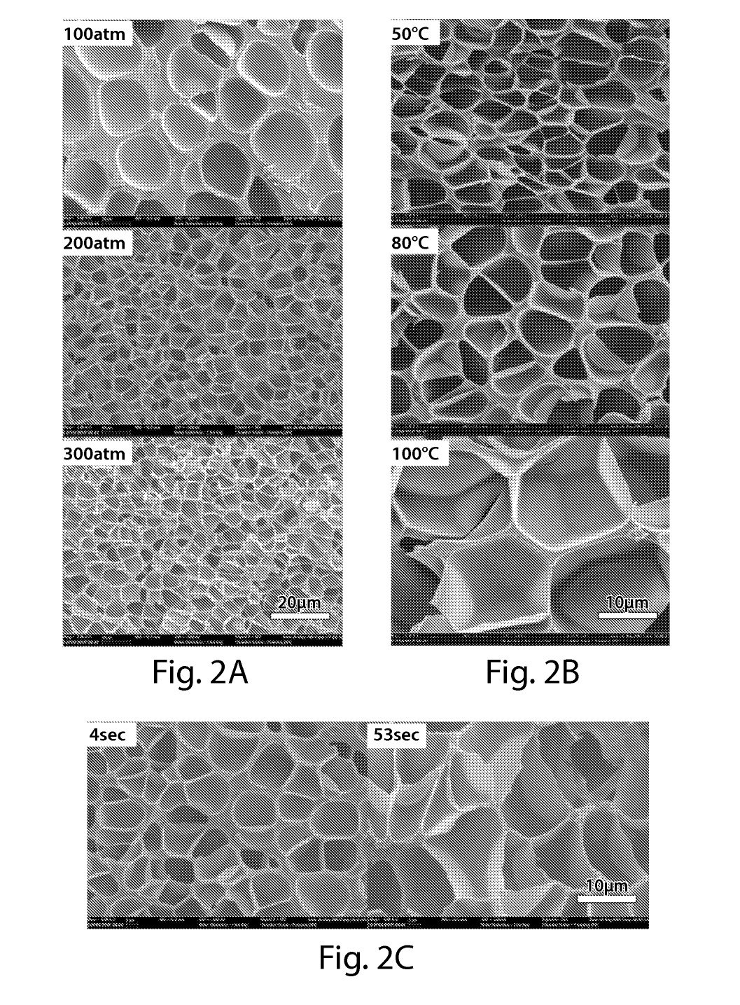

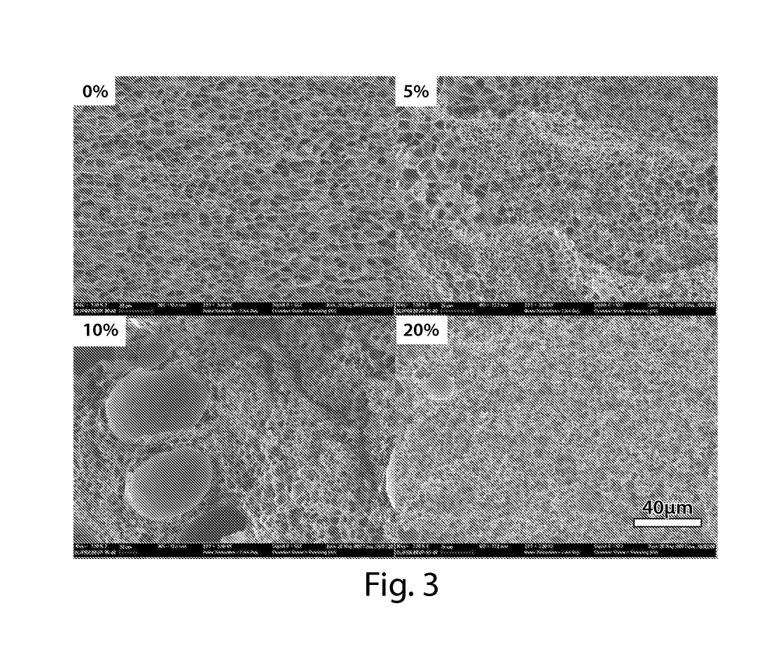

One aspect of the invention is generally directed to a foam that contains a pharmaceutically active agent, including techniques for creating such foams. As discussed below, in some embodiments, the foam has a relatively high specific surface area. The foam may be created using a supercritical fluid, such as supercritical CO₂, as is discussed below. Typically, the foam will include a pharmaceutically acceptable polymeric carrier, a pharmaceutically active agent in combination with the carrier, and “cells” or bubbles contained within the pharmaceutically acceptable polymeric carrier. The cells may contain a gas, such as CO₂ or air. Non-limiting examples of such foam structures can be seen in FIGS. 2 and 3.

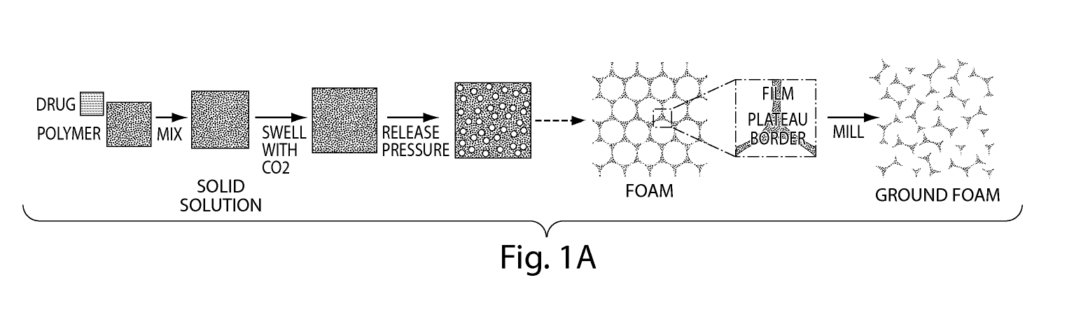

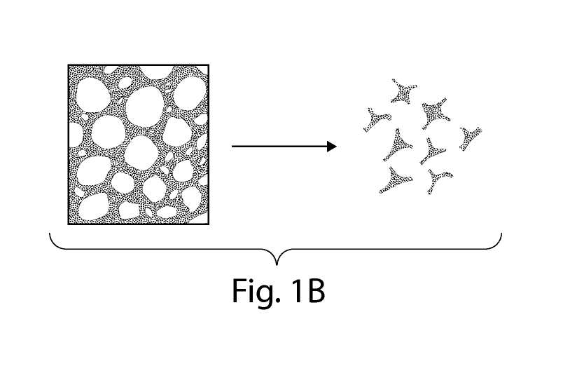

In some cases, a foam may be created by exposing a polymeric carrier to a foaming agent that can be dissolved or dispersed within the polymeric carrier at a first temperature or pressure, then by changing the temperature and/or pressure (in some cases, fairly rapidly), the foaming agent changes phase (e.g., into a gas), which causes bubbles or “cells” entirely surrounded by the polymeric carrier to form, thereby creating a foam structure in which the polymer forms a matrix surrounding empty regions, or “cells” therein. This can be seen in the schematic diagram of FIG. 1B on the left, where the foam structure includes a number of empty regions or cells therein. The cells may contain a gas, such as CO₂, air, or other foaming agents, or the cells may otherwise be substantially free of the polymer.

Examples of suitable polymers for use in the pharmaceutically acceptable polymeric carrier include, but are not limited to, poly(vinyl acetate) or poly(vinylpyrrolidone). In some cases, copolymers of these and/or other monomers may also be used, e.g., poly(vinylpyrrolidone-co-vinyl acetate) or polyvinyl alcohol-polyethylene glycol graft copolymer (for example, Kollicoat® IR from BASF). If a copolymer is used, the copolymer can have any suitable structure, such as a block copolymer, a random or statistic copolymer, an alternating copolymer, or the like. The copolymer may have 2, 3, or more monomers that define the copolymer. Any suitable ratio of monomers in the copolymer may be used. As a non-limiting example, if the copolymer includes vinylpyrrolidone and vinyl acetate, their ratio by weight may be about 6:4, about 4:3, about 1:1, about 2:1, about 3:1, about 10:1, about 1:2, about 1:3, about 1:10, or any other suitable ratio. The pharmaceutically acceptable polymeric carrier may comprise or consist essentially of one or more monomers such as those described herein.

The polymer within the pharmaceutically acceptable polymeric carrier can have any suitable molecular weight (also referred to as molar mass). For example, the molecular weight of the carrier may be at least about 10,000, at least about 20,000, at least about 30,000, at least about 50,000, at least about 70,000, at least about 100,000, at least about 200,000, or at least about 300,000. In some embodiments, the molecular weight may be no more than 500,000, no more than about 400,000, no more than about 300,000, no more than about 150,000, no more than about 100,000, no more than about 90,000, no more than 80,000, no more than about 70,000, no more than about 60,000, or no more than about 50,000. The molecular weight is often measured as a weight average molecular weight.

In some embodiments, the polymer is chosen to have a relatively high affinity for the foaming agent, for example, for CO₂. For example, at the operating pressure and temperature, the foaming agent may be soluble in the polymer at a concentration of at least about 10%, at least about 15%, at least about 20%, at least about 25%, or at least about 30% (determined on a weight basis), at least at Standard Temperature and Pressure (0° C. and 100 kPa or 1 bar). Foaming agents are discussed in more detail below.

The polymer within the pharmaceutically acceptable polymeric carrier may also be selected to be one which has a relatively low glass transition temperature (T_{g}), i.e., the temperature at which the polymer transitions from a relatively solid state to a more viscous or “rubbery” state, as is known by those of ordinary skill in the art. Glass transition temperatures can be determined using any suitable technique, for example, by measuring changes in viscosity, using DSC (differential scanning calorimetry), or the like. Typically, the polymer is foamed at a temperature above its glass transition temperature; however, temperatures that are too high may be detrimental to some types of pharmaceutically active agents. Accordingly, in certain embodiments, polymers having relatively low glass transition temperatures are used. For instance, the polymer may be one that exhibits a glass transition temperature of no more than about 200° C., about 180° C., about 160° C., about 150° C., about 140° C., about 130° C., about 120° C., about 110° C., about 100° C., about 90° C., about 80° C., about 70° C., about 60° C., about 50° C., about 40° C., or about 30° C. In some instances, the glass transition temperature is greater than about 0° C., about 10° C., about 20° C., about 30° C., about 40° C., about 50° C., about 60° C., about 70° C., about 80° C., about 90° C., or about 100° C. In one embodiment, the glass transition temperature is between about 95° C. and about 105° C. The polymer may be foamed at a temperature relatively close to its glass transition temperature in some embodiments. For example, the foaming temperature, i.e., the temperature of the polymer when the foaming process is initiated, such as by depressurization of the polymer, may be about 10° C., about 20° C., or about 30° C. above the glass transition temperature of the polymer.

The polymer may have any suitable material density. As used herein, the “material density” (also referred to as “bulk density”) of a polymer is the density of the polymer in the absence of any cells, foaming agents, or other non-polymeric materials (such as air or CO₂) trapped within the polymer. In contrast, the “foam density” of a foam is the overall mass of the foam divided by its volume, including anything trapped within the foam, such as a foaming agent. In certain embodiments, the polymer has a material density of less than about 3 g/cm³, less than about 2 g/cm³, less than about 1.5 g/cm³, less than about 1 g/cm³, less than about 0.8 g/cm³, or less than about 0.5 g/cm³. In some cases, the foam has a foam density of less than about 3 g/cm³, less than about 2 g/cm³, less than about 1.5 g/cm³, less than about 1 g/cm³, less than about 0.8 g/cm³, or less than about 0.5 g/cm³. It should be noted that the foam density is typically lower than the material density for a given foam.

In some embodiments, the polymer within the pharmaceutically acceptable polymeric carrier is a pharmaceutically acceptable polymer. For instance, the polymer may be bio-inert, biocompatible, or biodegradable. As used herein, “biocompatible” is given its ordinary meaning in the art. For instance, a biocompatible material may be one that is suitable for administration to a subject without adverse consequences. The pharmaceutically acceptable polymer may be one that can be swallowed by the subject, and the polymer may be relatively inert and pass through the subject without absorption or adverse consequences, and/or the polymer may be one that is degraded within the subject (i.e., the polymer may be biodegradable), and the products of degradation do not adversely affect the subject. For example, the biodegradable polymer may be one that is water soluble. Examples of biodegradable polymers include, but are not limited to, poly(caprolactone), poly(glycolic acid), poly(lactic acid), poly(3-hydroxybutyrate), etc., as well as copolymers of any of these and/or other suitable monomers. One non-limiting example is poly(lactic acid-co-glycolic acid).

In certain cases, the polymer within the pharmaceutically acceptable polymeric carrier is selected such that the polymer is water soluble. The water-soluble polymer may exhibit a reasonable rate of dissolution in water; for example, 10 g of the polymer may dissolve within 1 liter of water within less than one week, one day, 12 hours, or 3 hours, etc. For instance, upon administration to a subject, the polymer can begin to dissolve within the subject, thereby releasing the pharmaceutically active agent internally of the subject. In some cases, the rate of dissolution of the polymer may be controlled, e.g., by adding one or more monomers to the polymer that slow dissolution, and/or by controlling the monomers or the monomer ratios within the polymer in order to achieve a desired dissolution speed. As a specific example, dissolution speed may be increased by copolymerizing a relatively fast-dissolving monomer, such as lactic acid, or dissolution speed may be decreased by copolymerizing a relatively slow-dissolving monomer, such as glycolic acid.

As mentioned, a foam typically includes a pharmaceutically acceptable polymeric carrier, e.g., as described above, that contains bubbles or “cells” entirely surrounded by the polymeric carrier. According to certain aspects of the invention, the foam has an unexpectedly high specific surface area. Such a high specific surface area may, in some cases, facilitate delivery or release of the pharmaceutically active agent. For example, the foam can be milled to expose the internal surfaces of the foam, and the resulting milled particles are administered to a subject. In comparison with other foams having similar masses, formed using similar techniques (e.g., using supercritical CO₂ as discussed below), and carrying pharmaceutically active agents at relatively high loadings (e.g., at loadings of at least about 5 wt % based on the weight of the foam), the foams as discussed herein have much higher specific surface areas than would be expected for such foams created under such conditions. Without wishing to be bound by any theory, it is believed that such unexpectedly high specific surface areas are the result of surprisingly high cellular number densities and small cell sizes (e.g., microcellular foams), which are created by creating well-homogenized precursors and subjecting the precursors to rapid changes in pressure and/or temperature, as is discussed in detail below. In one set of embodiments, the foam is a “blown foam,” i.e., a foam formed by mixing or injecting a gas into a liquid, and causing the mixture to solidify to form the final foam.

As used herein, the “specific surface area” is a measure of the total surface area of the foam (both externally and internally, i.e., within the cells) per unit mass of the foam. The mass of the foaming agent within the foam is typically negligible relative to the mass of the polymeric carrier, especially if the foaming agent is a gas that is contained or trapped within the foam, and/or if the foaming agent is able to leave the foam after formation, often being replaced by air.

The specific surface area can be determined using any suitable technique. For example, the specific surface area can be determined using BET once the foam is milled to expose the internal surface area, or the specific surface area can be estimated using the average cell size, the volume fraction of the cells, and the density of the polymer forming the foam (see Example 1 for an example of this). In some cases, e.g., if the foam has closed cells, the foam may be ground prior to determining the surface area. The foam can have, in various embodiments, a specific surface area of at least about 0.1 m²/g, at least about 0.2 m²/g, at least about 0.3 m²/g, at least about 0.4 m²/g, at least about 0.5 m²/g, at least about 0.6 m²/g, at least about 0.7 m²/g, at least about 0.8 m²/g, at least about 0.9 m²/g, at least about 1 m²/g, at least about 2 m²/g, at least about 3 m²/g, at least about 4 m²/g, at least about 5 m²/g, at least about 6 m²/g, at least about 7 m²/g, at least about 8 m²/g, at least about 9 m²/g, at least about 10 m²/g, at least about 12 m²/g, at least about 15 m²/g, at least about 20 m²/g, at least about 25 m²/g, at least about 30 m²/g, at least about 35 m²/g, at least about 40 m²/g, etc.

The cells may have any shape or size within the foam, and may also have any size distribution. In some cases, the foam has an average cell size of less than about 10 micrometers. While cells can vary in shape and/or size, an average cell size can be defined as the average of the characteristic cell size for each cell within the foam, where the characteristic cell size for a cell is the diameter of a perfect sphere having a volume equal to the volume of the cell. Typically, such dimensions are estimated, e.g., from SEM (scanning electron microscopy) images, TEM (transmission electron microcopy) images or the like, rather than being precisely calculated, due to the heterogeneous distribution of cell shapes and/or sizes within a typical foam. For instance, by examining a suitable number of SEM or TEM images of a foam (e.g., chosen from representative locations within the foam) to determine typical dimensions for the cells within each image, the average cell size within the foam may be determined.

The foam can have, in various embodiments, an average cell size of less than about 5 micrometers, less than about 4 micrometers, less than about 3 micrometers, less than about 2 micrometers, less than about 1 micrometers, less than about 0.5 micrometers, less than about 0.3 micrometers, or less than about 0.1 micrometers in some cases. In some cases, the average cell size may be greater than about 10 nm, greater than about 100 nm, or greater than about 1 micrometer. In another set of embodiments, the foam may have a void fraction of at least about 50 vol %, at least about 60 vol %, at least about 70 vol %, at least about 75 vol %, at least about 80 vol %, at least about 85 vol %, at least about 90 vol %, etc., where the void fraction is the volume of cells or bubbles in the foam, as compared to the total volume of the foam, i.e., the fraction of the foam that is defined by the cells or bubbles. In some cases, the void fraction is less than about 90 vol %, less than about 70 vol %, or less than about 50 vol %.

In certain embodiments the foam can be described as a “microcellular foam,” i.e., having an average cell size of less than about 100 micrometers, and in some cases, the average cell size may be less than about 10 micrometers, less than about 5 micrometers, less than about 3 micrometers, or less than about 1 micrometer. In some cases, the microcellular foam may have an average cell size of between about 0.1 micrometers and about 100 micrometers, or between about 0.1 micrometers and about 10 micrometers.

In some cases, the number density of the cells contained within the foam may also be determined. The number density of cells in a foam is the number of cells per unit volume. Any suitable technique may be used to determine or estimate the number density, for example, SEM or TEM of a representative number of locations and/or images from the foam, depending on the specific application. For example, the foam may have a cellular number density of at least about 10⁷ cm⁻³, at least about 10⁸ cm⁻³, at least about 10⁹ cm⁻³, at least about 10¹⁰ cm⁻³, or at least about 10¹¹ cm³.

The pharmaceutically acceptable polymeric carrier forming the foam may also comprise a pharmaceutically active agent, according to another aspect. The pharmaceutically active agent may be present within the foam in any suitable amount or concentration, for instance, at a concentration high enough that, when administered to a typical subject, a beneficial or desirable effect is observed. For example, the pharmaceutically active agent can be admixed within the pharmaceutically acceptable polymeric carrier at an amount of at least about 5 wt % based on the weight of the foam. In some cases, the pharmaceutically active agent may be present at least about 10 wt %, at least about 15 wt %, at least about 20 wt %, at least about 25 wt %, at least about 30 wt %, at least about 40 wt %, at least about 50 wt %, at least about 60 wt %, or at least about 70 wt % in some cases.

Any suitable pharmaceutically active agent may be used. In some cases, the pharmaceutically active agent is one which is able to be dissolved and/or dispersed within the pharmaceutically acceptable polymeric carrier, e.g., as previously described. For example, a solid solution of a pharmaceutically active agent in a pharmaceutically acceptable polymeric carrier may be formed in some cases, which means that, in certain embodiments, the agent may be homogenously distributed within the carrier, although in other embodiments, their distribution need not be homogenous. In one set of embodiments, the pharmaceutically active agent is not miscible or soluble in water. For example, the pharmaceutically active agent may be incapable of dissolving in water at ambient temperature and pressure to a concentration of at least 1 g/l. In some cases, however, the pharmaceutically active agent is one that can be homogenously dispersed in water. Non-limiting examples of pharmaceutically active agents that may be present within the foam include carbamazepine, itraconazole, fenofibrate, cholesterol, or clotrimazole.

The foaming agent used to create the foam, according to one aspect, is selected to be dissolved or dispersed within a polymeric carrier at a first temperature or pressure to create the foam precursor. The foaming agent also can change phase, e.g., into a gas, at a second temperature or pressure that the polymeric carrier is exposed to (typically, both temperatures and/or pressures are selected so that the polymeric carrier and/or the pharmaceutically active agent do not substantially degrade). By causing the foaming agent to change phase within the precursor, pockets or “cells” are formed by the foaming agent within the precursor, which creates the final foam structure. Accordingly, the foaming agent may be any suitable agent that can be dissolved or dispersed within the polymeric carrier at a first concentration at a first temperature or pressure, but is dissolved or dispersed within the polymeric carrier at a second temperature or pressure at a second concentration that is substantially lower than the first concentration. In some cases, the foaming agent may change phase between the first temperature or pressure, and the second temperature or pressure. For instance, the foaming agent may be dissolved or dispersed in the polymeric carrier at the first temperature or pressure, but may form a gas in the polymeric carrier at a second temperature or pressure. The size of the cells created by the foaming agent in the final foam may be a function of the homogeneity of the foaming agent within the precursor to the foam, and/or the rate at which the pressure and/or temperature is changed from the first pressure and/or temperature to the second pressure and/or temperature. In one set of embodiments, the foam is created in a “batch” process.

As a specific example, the foaming agent may be a gas at Standard Temperature and Pressure (0° C. and 100 kPa or 1 bar). When mixed with the pharmaceutically acceptable polymeric carrier, the foaming agent may become dissolved or dispersed therein. For example, as discussed in detail below, the foaming agent can be subjected to temperatures and/or pressures such that the foaming agent is not gaseous and can be dissolved or dispersed within the pharmaceutically acceptable polymeric carrier, before foaming, to create a foam precursor. The precursor may then be subjected to a change in pressure and/or temperature that causes the foaming agent, or at least a portion of the foaming agent within the precursor, to form a gaseous state. For instance, the change in pressure and/or temperature may cause a drop in the amount of foaming agent dissolved or dispersed within the precursor, which then can result in a change of shape, or bubble or cell formation within the precursor.

Examples of suitable foaming agents include, but are not limited to carbon dioxide, alkanes such as pentane or hexane, nitrogen, nitrous oxide, or chlorofluorocarbons including hydrochlorofluorocarbons, or mixtures thereof. Other examples include, but are not limited to, CCl₃F or CCl₂F₂.

In certain embodiments, the foaming agent, when dissolved or dispersed in a pharmaceutically acceptable polymeric carrier to create a foam precursor prior to foaming, may be exposed to pressures and temperatures that cause the foaming agent to be in a supercritical state, i.e., the pressure and temperature of the foaming agent, when contacted with the pharmaceutically acceptable polymeric carrier, are each greater than the critical pressure and the critical temperature for that foaming agent. In some cases, the use of supercritical foaming agents may be advantageous since a higher concentration of foaming agent may be dissolved and/or dispersed in the pharmaceutically acceptable polymeric carrier, relative to non-supercritical conditions. Accordingly, because of the higher concentration, greater foaming may be produced, e.g., resulting in a higher volume fraction of the cells and/or higher specific surface area of the resulting foam.

In one aspect, a foam may be created by exposing a pharmaceutically acceptable polymeric carrier to a foaming agent to form a precursor. The pharmaceutically acceptable polymeric carrier can also contain a pharmaceutically active agent. For example, a pharmaceutically acceptable polymeric carrier and a pharmaceutically active agent may be mixed together, then the mixture exposed to a foaming agent, forming a precursor. The precursor may then be subjected to a change in pressure and/or temperature which causes the foaming agent to form a gas, thereby causing the formation of cells within the precursor (containing both the pharmaceutically active agent and the pharmaceutically acceptable polymeric carrier), forming the foam.

In one set of embodiments, a pharmaceutically acceptable polymeric carrier and a pharmaceutically active agent are first mixed together. In some cases, they are mixed together to form a homogenous mixture, e.g., a molecular solution of the agent in the carrier. The pharmaceutically acceptable polymeric carrier and the pharmaceutically active agent may each be in any suitable phase (e.g., solid or liquid), and the mixture may also be, for example, a liquid mixture or a solid mixture. For example, the pharmaceutically acceptable polymeric carrier and the pharmaceutically active agent may be mixed together to form a solid solution or other solid mixture. In some cases, a solid solution so formed can be identified as being nearly homogeneous or transparent, for example, without any inclusions or dispersed phases therein.

The pharmaceutically acceptable polymeric carrier and the pharmaceutically active agent may be mixed together directly, or a cosolvent may be used to prepare the mixture. A cosolvent is a material in which the pharmaceutically acceptable polymeric carrier and the pharmaceutically active agent are each mixed with, e.g., dissolved or dispersed, and the cosolvent is then removed, leaving behind a homogenous mixture, such as a solid solution. A cosolvent can be selected such that each of the pharmaceutically acceptable polymeric carrier and the pharmaceutically active agent is able to be dissolved or dispersed within the cosolvent. The specific cosolvent selected may thus be a function of the pharmaceutically acceptable polymeric carrier and the pharmaceutically active agent, and the cosolvent may be water-soluble or water-insoluble, depending on the physical properties of the pharmaceutically acceptable polymeric carrier and the pharmaceutically active agent. For example, if the pharmaceutically acceptable polymeric carrier is poly(vinylpyrrolidone-co-vinyl acetate) and the pharmaceutically active agent is itraconazole, tetrahydrofuran is an example of a cosolvent that can be used. In some cases, the cosolvent may subsequently be removed, e.g., resulting in a powder or a solid which is a homogenous mixture of the pharmaceutically acceptable polymeric carrier and the pharmaceutically active agent. For example, the mixture may be dried or the cosolvent may be partially or completely removed by evaporation and/or heating of the mixture. As another example, the pharmaceutically acceptable polymeric carrier and the pharmaceutically active agent may be mixed together using melt extrusion techniques.

Solid mixtures formed as discussed above may, in some cases, be prepared or processed by milling or grinding the solid mixture to form a powder. For example, techniques such as milling, ball milling, cryomilling, compression, impacting, rollers, crushers, and the like may be used to prepare the solid mixture as a suitable powder. For instance, the solid mixture may be milled using any suitable technique (e.g., ball milling or planetary milling) to form a powder having particle sizes of less than about 1 mm, less than about 500 micrometers, less than about 300 micrometers, less than about 100 micrometers, less than about 50 micrometers, less than about 30 micrometers, less than about 10 micrometers, etc. Smaller particles sizes may be useful, for example, in removing a cosolvent, in promoting more rapid mixing with the foaming agent, etc.

In some cases, the powder may be pressed into pellets or tablets. Such pressing may be useful, e.g., to drive out any gases that may be trapped within the powder matrix, which could adversely affect foaming. Any suitable pressure may be used to press the powder, for example, at least about 1,000 lb/in², at least about 2,000 lb/in², at least about 3,000 lb/in², at least about 4,000 lb/in², at least about 5,000 lb/in², at least about 8,000 lb/in², at least about 10,000 lb/in², etc. (1 lb/in² is about 6.894757 kPa.) Any suitable press, such as a hydraulic press, may be used. The pressure may be applied, in one set of embodiments, until no more creeping is observed in the powder, i.e., such that no more movement or deformation is observed in the powder while pressure is being applied to it. In some cases, an elevated temperature may also be used to facilitate this process, for example, a temperature of at least about 50° C., a temperature of at least about 80° C., a temperature of at least about 100° C., a temperature of at least about 110° C., a temperature of at least about 120° C., etc. For instance, the solid mixture can be exposed to a temperature of between about 90° C. and about 110° C. The solid mixture may be heated before, during, and/or after pressing.

The solid mixture, e.g., formed as a powder or a tablet, etc., can then be exposed to a foaming agent to form a final precursor, which is then processed to form the final foam. In one set of embodiments, the precursor is formed under temperatures and pressures under which the foaming agent is able to be dissolved or dispersed within the solid mixture. For example, the foaming agent may be a gas, a liquid, a solid, or a supercritical fluid. In some cases, after formation of the solid mixture, the solid mixture may be allowed to “soak” the foaming agent into the solid mixture.

As a specific example, in one set of embodiments, the foaming agent is added under conditions in which the foaming agent is supercritical. The exact temperature and pressure used may vary depending on the foaming agent and its critical point. For instance, the temperature at which the foaming agent is added may be at least about 30° C. or at least about 35° C., etc., and/or the pressure at which the foaming agent is added may be at least about 50 atm, at least about 70 atm, at least about 100 atm, at least about 150 atm, at least about 200 atm, at least about 300 atm, at least about 400 atm, at least about 500 atm, etc. As a specific example, the foaming agent may be added at a temperature of between about 30° C. and about 50° C. and a pressure of between about 300 atm and about 500 atm, which are each greater than the supercritical point of CO₂. As another example, the pressure may be between about 350 atm and about 450 atm.

In some cases, the foaming agent may be mixed in the precursor such that the foaming agent forms at least about 5% by weight of the precursor. The foaming agent may also form at least about 10% by weight, at least 15% by weight, at least about 20% by weight, at least about 25% by weight, at least about 30% by weight, at least about 35% by weight, at least about 40% by weight, at least about 45% by weight, at least about 50% by weight, etc., of the precursor.

After formation, the precursor may be caused to form a foam by subjecting the precursor to a change in pressure and/or temperature which causes the foaming agent to form a gas. The exact pressure and/or temperature at which the foaming agent forms a gas may vary depending on the foaming agent. In some embodiments, the precursor may be exposed to ambient (atmospheric) conditions to cause foaming to occur, e.g., about 25° C. and about 1 atm (the actual conditions may vary somewhat). For example, the precursor may be kept in a sealed vessel having a controlled temperature and/or pressure, then the precursor exposed to the ambient environment, e.g., by opening a valve or port in the vessel to the external atmosphere. In other embodiments, the precursor may be exposed to suitable controlled conditions, e.g., having lower temperatures and/or pressures sufficient to cause the foaming agent to form a gas.

In some cases, the decrease in pressure to form a foam may be very rapid. More rapid depressurization rates may affect nucleation rate, which can lead to smaller cells in the final foam. For instance, the change in pressure may occur for a time of less than about 1 s, less than about 500 ms, less than about 250 ms, less than about 200 ms, less than about 150 ms, less than about 100 ms, etc. As a specific example, the change in pressure may occur for a time of between about 100 ms and about 200 ms.

In another aspect, the foam may be ground or milled, or otherwise processed to form particles, including nanoparticles. The particles may have any shape and size, and in some embodiments, these are determined by the initial foam. For instance, a foam containing cells may be broken up to produce discrete particles, where at least a portion of the shape of the particles is determined by the “cells” that were defined in the original foam. Such characteristic shapes may be readily identified by those of ordinary skill in the art, for example, in examining SEM or TEM images. In some embodiments, the particles may have an average characteristic dimension of less than about 1 mm, and in some cases, less than about 500 micrometers, less than about 300 micrometers, less than about 100 micrometers, less than about 50 micrometers, less than about 30 micrometers, less than about 10 micrometers, less than about 5 micrometers, less than about 3 micrometers, less than about 1 micrometer, less than about 500 nm, less than 400 nm, less than about 300 nm, less than 200 nm, less than 150 nm, less than about 100 nm, less than about 75 nm, or less than about 50 nm in some cases. The “characteristic dimension” of a particle is the diameter of a perfect sphere having the same volume as the particle, and the average of a plurality of particles may be taken as the arithmetic average. In some embodiments, the average characteristic dimension of the particles may be estimated using TEM or SEM images, e.g., of a representative number of particles in a sample.

Techniques for converting a foam into particles or nanoparticles include, but are not limited to, grinding (e.g., mechanically), milling (e.g., ball milling, planetary milling, cryo-milling), crushing, compression, impacting, rollers, or the like. The duration the technique is applied can also be controlled, e.g., to control the shape and/or size of the particles thereby formed. For instance, longer milling times may result in smaller particles and/or particles having fewer or smaller concave surface regions or portions readily identifiable as cell portions.

In one set of embodiments, the particles have a relatively high surface area. Relatively high surface areas can be achieved in some embodiments since the initial material (e.g., foams) also had a relatively high surface area, and suitable grinding of such foams does not immediately result in perfectly spherical particles, but instead produces irregular forms. For example, the particles so produced may have, in various embodiments, a specific surface area of at least about 0.1 m²/g, at least about 0.2 m²/g, at least about 0.3 m²/g, at least about 0.4 m²/g, at least about 0.5 m²/g, at least about 0.6 m²/g, at least about 0.7 m²/g, at least about 0.8 m²/g, at least about 0.9 m²/g, at least about 1 m²/g, at least about 2 m²/g, at least about 3 m²/g, at least about 4 m²/g, at least about 5 m²/g, at least about 6 m²/g, at least about 7 m²/g, at least about 8 m²/g, at least about 9 m²/g, at least about 10 m²/g, at least about 12 m²/g, at least about 15 m²/g, at least about 20 m²/g, at least about 25 m²/g, at least about 30 m²/g, at least about 35 m²/g, at least about 40 m²/g, etc. In one set of embodiments, particle irregularity may be determined by measuring the average characteristic dimension and the surface area of the particles as a function of mass, and comparing that to the theoretical surface area of spherical particles having the same average characteristic dimension (i.e., diameter) with respect to the same mass basis. The particles of the present invention may have, for example, at least about 1.5 times, at least about 2 times, at least about 2.5 times, at least about 3 times, at least about 4 times, or at least about 5 times the surface area of the theoretical surface area of the spherical particles.

The irregularity or morphology of the particles may be determined using techniques such as electron microscopy (e.g., TEM or SEM). As mentioned, the particles may be created by grinding or milling a foam containing cells into discrete particles, and in some cases, at least a portion of the shape or surface of the particles is determined by the cells that were present in the original foam. In some cases, at least some of the particles will have concave surface regions, as identified using such techniques. Concave surface regions may be created when the materials surrounding or interstitially positioned between the cells or bubbles of the foam are isolated; the isolated solid materials still may retain some of the structure previously defined by the cells or bubbles, thereby retaining a concave surface region in at least one portion of the particle. See, e.g., the particle shapes shown in FIG. 1A as a non-limiting example. As an illustration, referring now to FIG. 1B, particles having such shapes may be formed from an initial foam, which is ground to form particles having one, two, or more concave surface regions. In some embodiments, at least about 20%, at least about 30%, at least about 40%, at least about 50%, at least about 60%, at least about 70%, at least about 80%, or at least about 90% of the surface regions of the particle may be defined by one, two, or more concave surface regions.

It should be understood that the particles need not all have the same shape, and in some cases, some of the particles may contain one or more concave surface regions while other particles do not contain readily identifiable concave surface regions, e.g., as can be determined using techniques such as TEM or SEM. However, in the population of particles, at least some of the particles will be identifiable as having one or more concave surface regions. For example, in a sample of particles, on the average, at least about 20% of the particles can be identified as having at least one concave surface region. In some cases, at least about 30%, at least about 40%, at least about 50%, at least about 60%, at least about 70%, at least about 80%, or at least about 90% of the particles may be identified as having one or more concave surface regions.

In some embodiments, at least some of the particles may contain more than one concave surface region. For instance, the particles may be formed at the intersection of two or more bubbles or cells in the original foam. In some cases, at least about 20% of the particles can be identified as having at least two concave surface regions, and in some embodiments, at least about 30%, at least about 40%, at least about 50%, at least about 60%, at least about 70%, at least about 80%, or at least about 90% of the particles may be identified as being “multi-concave,” i.e., having two or more concave surface regions.

U.S. Provisional Patent Application Ser. No. 61/160,040, filed Mar. 13, 2009, entitled “Systems and Methods of Templating Using Particles such as Colloidal Particles,” by Weitz, et al.; and PCT Patent Application Serial No. PCT/US2010/000748, entitled “Systems and Methods of Templating Using Particles such as Colloidal Particles,” filed Mar. 12, 2010, by Weitz et al. are each incorporated herein by reference in their entireties.

Also incorporated herein by reference in their entireties are U.S. Provisional Patent Application Ser. No. 61/347,062, filed May 21, 2010, entitled “Foams or Particles For applications Such as Drug Delivery,” by Ladavac, et al.; U.S. Provisional Patent Application Ser. No. 61/347,082, filed May 21, 2010, entitled “Foams Including Microcellular Foams Containing Colloidal Particulates,” by Ladavac, et al.; and a PCT application filed on even date herewith, entitled “Foams Including Microcellular Foams Containing Colloidal Particulates,” by Ladavac, et al.

The following examples are intended to illustrate certain embodiments of the present invention, but do not exemplify the full scope of the invention.

### Example 1

This example illustrates a process for making microcellular polymer foams containing active pharmaceutical ingredients (APIs). These foams can be ground to make small, irregularly shaped particles of API-laden polymer. The large surface area of these foams and particles improves the dissolution of the API in water, in particular its dissolution rate, and improves bioavailability. The process is well-suited for APIs with low solubility in water.

In this example, the polymer is foamed directly using high pressure supercritical CO₂, without any solvents or surfactants. The foam morphology is controlled by the applied pressure, operating temperature, and the pressure release rate. At appropriate combinations of these variables, microcellular foams with 3 micrometer pores at 85% volume fraction were produced. The API content could be varied from 0 to 20% by mass. Higher loading decreased the pore size and increased the surface area, which suggests that the API helps to nucleate bubbles in the foam. The dissolution of API from ground foam was also compared with that from a non-porous solid solution, and it was shown that the drug incorporated in a foam showed both an increase in the dissolution rate and apparent oversaturation.

This technique is general and can be extended to different polymers and APIs by tuning the operating parameters. For instance, three different polymers were successfully foamed, one in combination with two different APIs.

This example illustrates a technique to process APIs and enhance their bioavailability. Bioavailability describes both the extent and rate of absorption of an API, or a drug, by the human body. Bioavailability is therefore related to both the solubility and the rate of dissolution. More than about 40% of newly discovered drug candidates have little or no water solubility, and more than about 90% of drugs approved since 1995 have relatively poor solubility. The difficulty of delivering such hydrophobic drugs precludes their widespread use. Formulating a way to deliver poorly soluble drugs could not only improve the efficiency of existing drugs, but also boost the development of new ones.

Without wishing to be bound by any theory, the dissolution rate, as described by Nernst-Brunner modification of the Noyes-Whitney model, depends on the total surface area exposed to the dissolving medium:

$$\frac{d C}{d t} = \frac{DA}{Vl} ( {C}_{s} - C )$$

where C is the instantaneous concentration, Cₛ is the saturation concentration (solubility), D is the diffusivity, l is the thickness of the diffusion layer, V is the volume of the medium, and A is the area of the dissolving particle. To enhance the dissolution rate, and the bioavailability, a larger surface area per volume or, equivalently, reducing the particle size of the API is suggested by this equation.

This example illustrates a method to make API-laden particles with relatively high specific surface area. The API is incorporated into a dry polymer foam (with a high gas volume fraction), and the surface area is controlled by controlling the foam length scale. Some portion of the drug is contained in the “plateau borders” of the foam, where three or more adjacent cells of the foam come close to or into contact with each other (see FIG. 1A). The last step in this example is to grind the foam, which opens up at least some of the pores to expose the interior surface for drug release. In this process, by using a polymer foam rather than foaming the drug directly, no surfactants are used, many polymers that are good solvents for APIs can be selected, and the polymers may be solidified and ground into small particles. Due to the insufficient mechanical properties of pure APIs in the absence of a polymeric carrier, foams of APIs in the absence of a significant amount of a polymeric carrier cannot be stabilized, at least in some cases.

At high temperatures and pressures, supercritical carbon dioxide (SC CO₂) is a good solvent for certain polymers and is readily absorbed. The small CO₂ molecules may create more free volume for the polymer chains, thereby depressing the glass transition temperature, T_{g}. If the working temperature lies above the zero-pressure T_{g}, the pressurized sample may be liquid. A rapid pressure drop can lead to immediate phase separation, and the CO₂ bubbles can nucleate and grow. Also, if the working temperature lies between the high pressure and the zero-pressure T_{g}, as CO₂ leaves the polymer, the T_{g} may increase until it reaches and exceeds the working temperature, at which point the polymer becomes glassy, and the structure may be quenched.

A batch processing setup is used in this example, which includes a high pressure chamber that is pressurized through a pump and depressurized by opening a valve. Both the operating pressure and temperature were controlled, allowing parameters that are optimized for each combination of polymer and the API to be chosen. A high depressurization rate was used to achieve a high bubble nucleation density, which yielded small pores with larger surface areas.

This example shows that the presence of an API can reduce the bubble size, thus increasing the surface area exposed to the dissolving medium and improving bioavailability. Without wishing to be bound by any theory, it is believed that the APIs may be behaving as nucleating agents in this process. For instance, the bubble size was found to scale with the amount of API present, and appeared to be reproducible for both of the APIs used in this example.

Polymers and other materials used in this example included poly(vinyl acetate) (PVAc; Aldrich, CAS 9002-89-5; M_{w}˜85,000-124,000), poly(vinylpyrrolidone) (PVP; Aldrich, CAS 9003-39-8; M_{w}˜360,000), poly(1-vinylpyrrolidone-co-vinyl acetate) (PVPVA; Aldrich, CAS 25086-89-9; M_{w}˜50,000), cholesterol (Alfa Aesar, 96% pure, CAS 57-88-5); clotrimazole (BASF); tetrahydrofuran (THF; EMD, DriSolv, CAS 109-99-9); carbon dioxide (CO₂; Igo's Welding Supply, Coleman Grade, minimum purity 99.99%, liquid). High pressure foaming is a general approach that works for a variety of polymers. This example illustrates foams with several different polymers, including PVAc, PVPVA, and PVP. For the examples here, PVPVA was used as a model polymer because it has properties intermediate between those of PVAc and PVP.

Polyvinylacetate (PVAc) has a high affinity for CO₂. At 25° C. and a vapor pressure of ˜60 atm, the solubility of CO₂ in PVAc is about 30%. The glass transition temperature T_{g} of the polymer in the absence of CO₂ is about 28° C. to 30° C. The low working temperature and pressure make the polymer easy to foam. But the low T_{g} also means that PVAc may melt at body temperature when it contains APIs. Also, it is not water soluble. By contrast, PVP has high water solubility, but also a high glass transition temperature, around 180° C. The copolymer polyvinylpyrrolidone-co-vinylacetate (PVPVA) appeared to combine some of the better properties of PVP and PVAc. For instance, it is water soluble, the acetate groups provide binding sites for CO₂, which may increase absorption, T_{g} is about 100° C., and it is a good solvent for many APIs.

Before preparing the foam, a solid solution of the API in polymer was first produced. The polymer and the API were mixed with a cosolvent (for example, THF), and the cosolvent was then evaporated. The samples were first dried on a polyethylene sheet overnight at 50° C. under air, then milled for around 10 hours (Retsch Planetary Ball Mill PM 100) at 300 RPM in a 12 ml chamber with 4 stainless steel balls of 10 mm diameter. Then they were dried again at 50° C. overnight. The result was a powder containing both polymer and the API, with a grain size around 100 micrometers or smaller. The milling step thus appeared to make the drying process more efficient.

Prior to foaming, the powders were pressed into homogeneous bulk pellets with thickness of greater than 1 mm. This is because the foaming of a powder often leads to lower quality foams: near the surface of the polymer, gas diffuses out instead of nucleating bubbles, leading to a skin layer typically 30 to 40 micrometers thick. The skin layer, in some cases, prevented or reduced foaming in this region. Thus, in some experiments, the surface area of the solid solution prior to making the foams was reduced by pressing the powder grains together.

A hydraulic press (Harco Industries) with an 1 inch inner diameter round steel die wrapped with a silicone-rubber heat sheet (McMaster-Carr, 6 inch×6 inch (15 cm×15 cm) which was temperature controlled using a PID controller (Omega Engineering, CSI32K iSeries Benchtop controller) that maintained the working temperature by a feedback loop through a thermocouple (Omega, KHSS-18G-RSC) was used. The powder was first pressed (7,000 pounds of force, or about 31 kN) to reduce the amount of air and therefore prevent any oxidation. The sample was pressed until it stopped creeping, which may be due to most of the air being evacuated. The sample was then heated to about 100° C. Because increasing the mechanical pressure may increase the glass transition temperature, the pressure was reduced to about 100 psi (about 690 kPa) to let the powder flow and fully sinter. The sample was left in the press for a few hours. The final mixtures were clear and transparent, which suggested a solid solution of API in polymer.

Foaming with supercritical CO₂ required initially creating CO₂ bubbles (e.g., through nucleation), then quenching the structure. The quench happens when gas leaves the polymer matrix, thus shifting the T_{g} above the working temperature. This process can be viewed as a double phase transition: first CO₂ separates from the solution to form bubbles, then the solution itself turns into a glass. Several operating parameters therefore need to be controlled, including temperature, pressure, the depressurization rate, and the soak time.

The high-pressure setup used in this example included a CO₂ cylinder, pump, chamber, and assorted valves and fittings. Gas was drawn from the cylinder to a high pressure syringe pump, model 260D from Teledyne Isco (Lincoln, Nebr.). The pump capacity was 266 ml, and the maximum pressure was about 7500 psi (about 52 MPa). Samples were foamed in a 100 ml hand-tight steel chamber made by Pressure Products Industries Inc. (Warminster Pa.), purchased from Supercritical Fluid Technologies Inc. (Newark Del.).

To keep the polymer and the API from oxidizing or reacting at elevated temperatures, air was purged from the chamber immediately after loading the sample by flushing the chamber a few times with low pressure CO₂. The chamber was then heated to the target temperature. Once it reached the target temperature setpoint, the target pressure was increased by pumping in CO₂. The sample was left to soak for a given time at constant temperature and pressure, and then the pressure was released by opening the valve to the atmosphere.

This setup allowed operating temperatures up to 200° C. and pressures to about 7,300 psi (about 50 MPa). The lower bound appeared to be the critical point of CO₂, 31.1° C. and 1,080 psi (7.4 MPa). It is possible, however, to work below the critical temperature, although the applied pressure may be limited in some cases by the vapor pressure of the liquid CO₂. The depressurization rate was controlled since it can affect the nucleation rate. Higher depressurization rates were found to be better, as they lead to higher nucleation rates, smaller bubbles, and smaller length scales. The depressurization time was reduced by reducing the dead volume in the chamber. The shortest time achieved was on the order of few seconds.

In experiments where APIs were added to the polymer, in some cases, some pure API was added to the chamber prior to foaming. This was to saturate the CO₂ with the API and prevent the API in the precursor from dissolving into the fluid. For example, cholesterol has a high solubility in CO₂, about 2 g/l at typical operating pressures and temperatures, and loading prior to foaming appeared to produce better results.

Scanning electron microscopy (SEM) was used to image the foam structure. To make samples for imaging, the foamed samples were first cut opened by freezing them in liquid nitrogen to make them brittle, then fracturing them with a sharp blade. The fractured pieces were sputter-coated with a ˜10 nm layer of platinum to keep the samples from charging under the electron beam. A Zeiss Supra55VP SEM (Harvard Center for Nanoscale Systems) was used for imaging.

Prior to dissolving the polymer, the foam was ground to open up the closed bubbles and expose as much surface area as possible to the solvent. The goal was not necessarily to achieve flakes of single films or plateau borders, since powder grains of a few bubbles should still have a relatively increased surface-to-mass area. A Retsch Planetary Ball Mill PM 100 with a 12 ml chamber containing four 10 mm stainless steel balls was used. All samples were ground under the same conditions. To avoid heating and possibly melting samples, the mill was run at 300 RPM at 50% duty cycle (1 minute on/1 minute off) for a total of 20 hours.

Dissolution tests were performed at 37° C. using a method similar to the USP paddle method. Briefly, the stiffing speed was 100 RPM, and distilled water (pH of 7) was used as the solvent. Microcrystalline cellulose (20 micrometer powder, Aldrich 310697) was added so that the grains did not clump up as the polymer swelled in water. The foam was directly milled with cellulose, whereas the corresponding (unfoamed) solid solution was milled first, before adding cellulose. Without cellulose, the dissolution rate was determined by how fast the polymer leached away from the surface of the clump, and not by dissolution from the surface of individual powder grains. In a typical experiment, 100 mg of formulation (e.g., 50 mg cellulose with 50 mg foam, containing 10 mg clotrimazole) was added to 300 ml water. The powder wetted quickly and was completely submerged within the first 10-20 seconds in all cases (both with foamed and unfoamed solid solutions).

The chamber was directly connected to a spectrometer (Perkin Elmer Lambda 40) through a peristaltic pump. The bottom of the chamber was fitted with a 0.45 micrometer pore filter that prevented any undissolved particles from reaching the spectrometer. The solution flowed through continuously, with a delay time of less than one minute between the chamber and spectrometer. After the spectrometer, it was recirculated back to the chamber keeping the total volume constant. The amount of API used was above the saturation concentration, assuring both the dissolution rate and saturation could be observed. The relative absorbance was used at 262 nm as a measure of clotrimazole content.

Effects of pressure, temperature and depressurization rate on foam morphology were as follows. The effect of operating variables on the structure of pure PVPVA foams was studied first. It was found that increasing the operating pressure resulted in drier foams. FIG. 2A illustrates how the foam morphology changed with applied pressure (P). In this figure, P was 200 atm, the temperature was 50° C., the soak time was 2 to 3 hours, and the depressurization time was 2 to 4 s. Without wishing to be bound by any theory, it appears that the reason for the change in morphology is that the density of the supercritical fluid was higher at higher pressure. From 100 atm to 200 atm the supercritical CO₂ density doubled, and the resulting foam was substantially drier. Going to 300 atm improved the foam further, although to a lesser degree, as the fluid density increased by only about 10%.

The effect of temperature is shown in FIG. 2B, using experimental conditions similar to that shown in FIG. 2A. At lower temperatures, the bubble number density was higher. This again may be an effect of the supercritical fluid density: at lower temperature, the fluid density was higher. Because the solubility of CO₂ in polymers increased with density, the thermodynamic instability upon pressure release may be stronger, and as a result the nucleation rate may increase. This mechanism may lead to higher final bubble volume fractions and number densities, which may thus result in thinner films and plateau borders. Also, Ostwald ripening leading to an increase of bubble size is reduced at lower temperature.

There may also be a limit to working with lower temperatures. Changing the temperature also influences how quickly the polymer structure quenches to “freeze in” the bubbles. For instance, at lower temperatures, the foam may have less time to dry before the polymer vitrifies, as seen in FIG. 2B. 50° C. foams were less dry than ones processed at 100° C., where the films are of comparable thickness to the plateau borders. Thus, there may be more time for the polymer to flow at 100° C.

To examine the influence of pressure release rate, two samples were foamed at the same pressure, temperature, and different depressurization times. For the fast pressure drop, the valve was opened fully in the shortest time possible (4 s). In contrast, for the slow pressure drop, the valve was opened midway, then slowly (over 53 s) opened up more as the pressure inside the chamber dropped. Although this precision is coarse, the experiments revealed a qualitative difference between the two depressurization rates, as shown in FIG. 2C. In this figure, fast depressurization resulted in a higher nucleation rate, with higher bubble number density and smaller bubble size. Faster depressurization also yielded a faster quench. The sizes of the films and plateau borders appeared to be heterogeneous, suggesting that there was not enough time for polymer to flow.

The effect of API loading on PVPVA foams was studied next. To make API-laden polymer foams, a model API was added to the polymer before foaming. Clotrimazole and cholesterol were used in this example as model APIs. PVPVA was loaded with the API at concentrations ranging from 0% by mass to 30% by mass, as described above. The solubility of APIs in PVPVA was first tested by dissolving both the API and the polymer in a solvent, then spreading a thin layer on a glass slide to dry. With cholesterol, crystals were observed to precipitate at about 30% loading. These crystals were visible under bright-field and cross-polarized optical microscopy. At concentrations of 20% and lower, the films appeared to be fully transparent, suggesting a solid solution. Solid solutions of clotrimazole were obtained at up to 20% loading.

It was found that the APIs had a beneficial effect on the foam morphology: they appeared to make the bubble size smaller and the films and plateau borders thinner. Cross sections of PVPVA foams with different cholesterol loading are shown in FIG. 3. These were prepared simultaneously in the high pressure chamber, so that they were all subject to the same temperature, pressure, and depressurization rate. The cholesterol weight fraction is indicated on each image, and the scale bar is the same for all images. The foams were prepared at 200 atm and 50° C., and the foams were soaked for 4 hours with a depressurization time of 4 s.

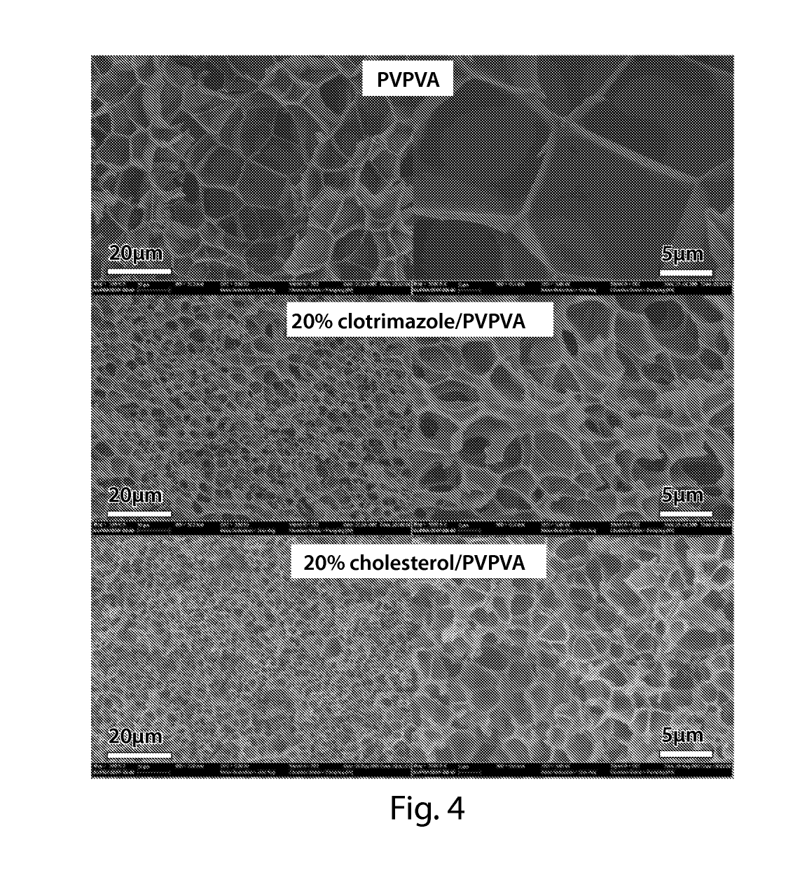

Without an API, the pure polymer foam appeared homogeneous, with monodisperse bubbles that were uniformly distributed throughout the sample bulk. But with cholesterol, a second length scale appears. At 5% loading, the bubble size distribution appeared bidisperse, with different size bubbles appearing in different regions. The size of the large bubbles was the same as that in the pure polymer case, but there was also a population of bubbles with an average diameter of about ⅓ that of the large bubbles. The number density of the smaller bubbles increased with the API loading, which may suggest that the API helped to nucleate bubbles. Similar results were seen with a second API, clotrimazole (20%), as shown in FIG. 4. The foaming conditions were the same in each case: 200 atm and 50° C., with a depressurization time of 4 s. For both APIs, the size of the smallest bubbles was significantly smaller than those for pure polymer foams.

Without wishing to be bound by any theory, the specific surface area from the bubble size and volume fraction can be estimated as follows:

$$\frac{S}{m} = \frac{4 π {r}^{2}}{( ( 4 / 3 ) π {r}^{3} ) ρ ( 1 - ϕ )} = \frac{3}{ρ ( 1 - ϕ ) r}$$

Here S is the surface area, m is the mass, r is the bubble radius, ρ(rho) is the density of API-laden polymer, and φ(phi) is the bubble volume fraction. This relation gives only a lower bound on the total surface area because it assumes that the bubbles are spherical, closed, and undeformed.

For the 20% clotrimazole/PVPVA foam presented in FIG. 4, the specific surface area was estimated at 10 m²/g, equivalent to the surface area of monodisperse spherical particles of 500 nm diameter. This is based on an estimate of the average bubble diameter (3 to 4 micrometers) from the SEM images, and a measurement of the sample volume before and after foaming, which allows the volume fraction (φ) to be estimated at 0.85.

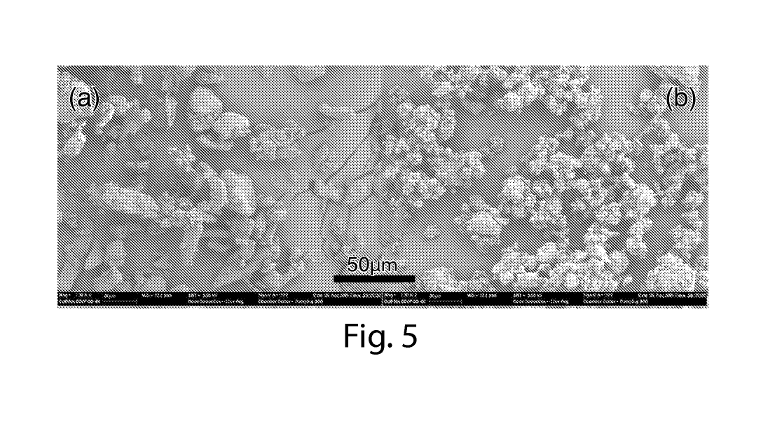

Next, the dissolution behavior of the API-laden foams discussed above was compared with a solid solution. Before dissolving, both samples were ground at the same speed and for the same amount of time. The left image in FIG. 5 is a ground solid solution, while the right image in FIG. 5 is a ground foam. It was found that the resulting powders had similar grain sizes. This was expected, as the grain size can be controlled by the grinding conditions. But the grains made from the above-described foams appeared to have a different morphology: they were fractal-like, flaky, and irregularly shaped, suggesting that they had higher surface area than the grains from the solid solution at comparable grain size.

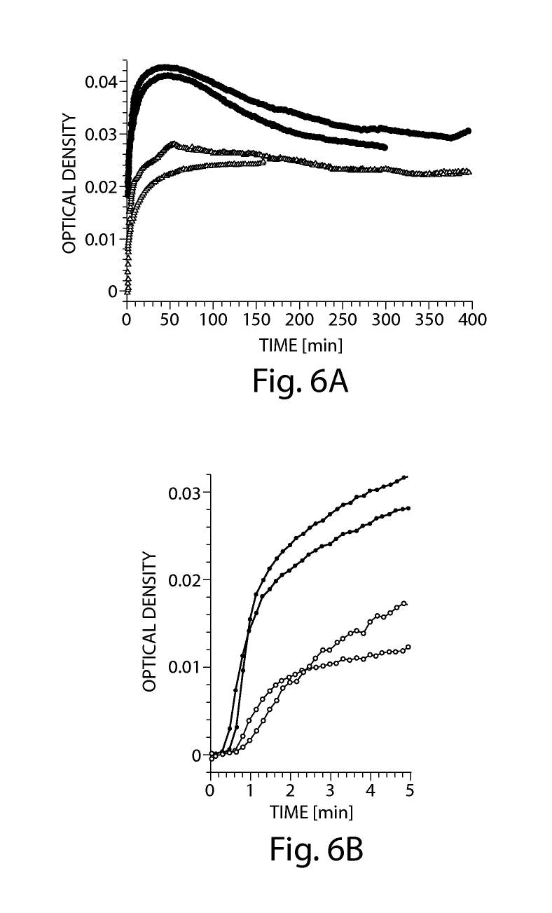

FIG. 6 illustrates data from the dissolution tests. The raw data from the instrument, referred to as optical density, is considered to be directly proportional to the concentration of dissolved clotrimazole. In FIG. 6A, the lower two lines are ground solid solution, while the upper two lines are ground foam. FIG. 6B shows the same data, except zoomed at early times. The ground foam had a higher dissolution rate (FIG. 6B) and a higher apparent oversaturation after about 50 minutes than the ground solid solution. The dissolution tests were performed twice for each sample, and the same results found both times.

The increase in the dissolution rate suggests that the ground foams had a higher specific surface area than the ground solid solutions. This increase in surface area may be due to the interior morphology of the particles, which is a result of the foaming process. It may be expected that the increased dissolution rate is independent of solvent because it is due to increased surface area. Therefore, the foam samples also may dissolve more quickly in gastrointestinal fluid, thereby increasing API bioavailability in vivo. Oversaturation as observed for the samples can also be beneficial for bioavailability.

In conclusion, this example illustrates a process to increase the dissolution of APIs with low water solubility. The APIs were incorporated into a solid polymer foam of controlled morphology. It was shown that bubble size, film and plateau border thickness can be optimized to achieve maximum surface area by judicious control of temperature, pressure, and depressurization rate. When the foam was ground, API-laden polymer particles could be obtained that dissolved faster and obtained higher oversaturation than ground solid solutions.

### Example 2

This example illustrates foams containing itraconazole, fenofibrate, and carbamazepine. The experimental high pressure setup used in this example was similar to the one used in Example 1, except a 3-way valve was added, which has a wider bore opening so gas can leave faster. It was actuated pneumatically, faster than hand-turning the ball valve as was the case in Example 1. In the “closed” position, the valve connects the chamber to the rest of the high pressure manifold (to the pressure gauge and pump) and closes it to atmosphere. In the “open” position, the chamber is open the atmosphere, but closed to the rest of the manifold. This reduces the volume that is vented (when the valve is opened), which substantially cuts down the pressure release time.

The use of higher pressure resulted in a higher supercritical fluid density, hence more CO₂ could be dissolved in the polymer. In this example, the applied pressure was 400 atm instead of 200 atm as used previously. Faster pressure release rates also may induce stronger thermodynamic instability, and nucleate more bubbles. Also, there is less time for the polymer to flow during the pressure release step. This allows operation under conditions that are further away from the glass transition, at lower temperatures, where fluid density is higher. The carrying polymer used in this example was the BASF brand of poly(1-vinylpyrrolidone-co-vinyl acetate). Compared to the polymer used in Example 1, this differed in the compositional ratio of vinyl pyrrolidone to vinyl acetate (now 6:4, instead of the Aldrich brand, which was 4:3 by mass). However, the solubilizing powers were similar, and the glass transition temperature of pure polymer was 110° C.

Most of these experiments were performed at 400 atm pressure, 40° C. temperature, and pressure release times of about 100-200 ms. The materials used were KVA64, poly(1-vinylpyrrolidone-co-vinyl acetate) (BASF, brand name Kollidon® VA64, M_{w}˜45,000-70,000), itraconazole (BASF), fenofibrate (BASF), carbamazepine (BASF), and CO₂ (Igo's Welding Supply, Coleman Grade, minimum purity 99.99% liquid phase).

Solid solutions of APIs in polymer were prepared by melt extrusion. A small scale twin screw extruder was used (Micro Compounder, DACA Instruments), courtesy of the Prof. Cohen Laboratory at MIT. Samples were extruded at temperatures just above the API's melting point. Sufficient mixing resulting in true solid solutions was confirmed by observation (optically clear extrudate, no crystals under SEM) and by DSC (no melting signatures, single glass transition). The extrudates were loaded in the high pressure chamber and foamed as described in Example 1.

The features in all the foams prepared in this example were smaller than before, suggesting that these operating parameters were improved. The bubble and foam dryness varied depending on the API. The thickness of films and plateau borders was on the order of 200 nm at most.

Various foams produced in this example include pure polymer foam prepared at 400 atm, 40° C., soaked for 2 hours, depressurization time below 1 s; and fenofibrate/polymer foams prepared at 400 atm, 40° C., soaked for 2 hours, depressurization time below 1 s. The API loading level was 10-30%. The presence of fenofibrate appeared to significantly lower the glass transition temperature: higher loaded foams had further time to flow before quenching and the resulting bubbles were larger. Additional foams produced in this example include 20% carbamazepine in polymer foams prepared at 400 atm, 40° C., soaked for 4 hours, with a depressurization time below 1 s; and itraconazole/polymer foams prepared at 400 atm, 40° C., soaked for 4 hours, depressurization time below 1 s. The API loading level was 10-20%.

### Example 3

Other polymers besides PVPVA may be used to prepare foams. For example, in this example, foams using polyvinylpyrrolidone (“PVP”) are demonstrated. In this example, PVP was obtained from BASF (Kollidon® 90F), and used to prepare polymer foams. The foaming conditions used in this instance to produce the polymer foams were an initial pressure of 300 atm at 160° C. with a soak time of 2 hours, followed by a 2 s depressurization time.

### Example 4

This example illustrates an approach for preparing drug formulations based on confinement rather than synthesis or milling. The premise is that the surface-to-volume ratio of any non-fractal object with smallest dimension h scales as 1/h, regardless of the shape of the object. For example, a large, thin film has a ratio of 2/h, versus 6/h for a sphere with diameter h. Thus it is possible to create high surface areas simply by shrinking the size of the material to the nanoscale in one dimension only.

To implement this idea, inclusions such as bubbles were embedded within a solid solution, then packed together to confine the domains of active material into thin films, as shown in FIG. 1A. Nanoscale films were made using relatively large inclusions; the inclusions can be packed together efficiently in some cases, well beyond the 74% volume fraction that can be achieved using close-packed spheres. To this end, bubbles were used as inclusions in this example. It was shown that these techniques can increase the dissolution rate of model, hydrophobic actives compared to the state-of-the-art pharmaceutical formulations. The approach is a simple and general route to nanostructured materials. Although shown here with respect to certain pharmaceutical actives, these techniques may be extended to other systems, for example, other actives, polymers, and the like.

In this example, high pressure CO₂ was used to grow and nucleate micrometer-scale gas bubbles in a solid solution, as shown in FIG. 1A. Certain hydrophobic drugs (“actives”) and a polymer that can dissolve greater than 20% w/w of any of the actives were used in this example. The resulting solid solution was exposed to high-pressure, supercritical CO₂ that swelled the polymer and created free volume between polymer coils, thus depressing the glass transition temperature (T_{g}) of the polymer. Choosing a working temperature between the high-pressure and atmospheric-pressure T_{g} ensured that the pressurized sample was liquid but vitrified when the pressure was released. After a soak time of few hours, the pressure was released, which lead to rapid phase separation and nucleation and growth of CO₂ bubbles. As CO₂ left the polymer, T_{g} increased. When it reached the working temperature, the polymer becomes glassy and the structure was quenched.

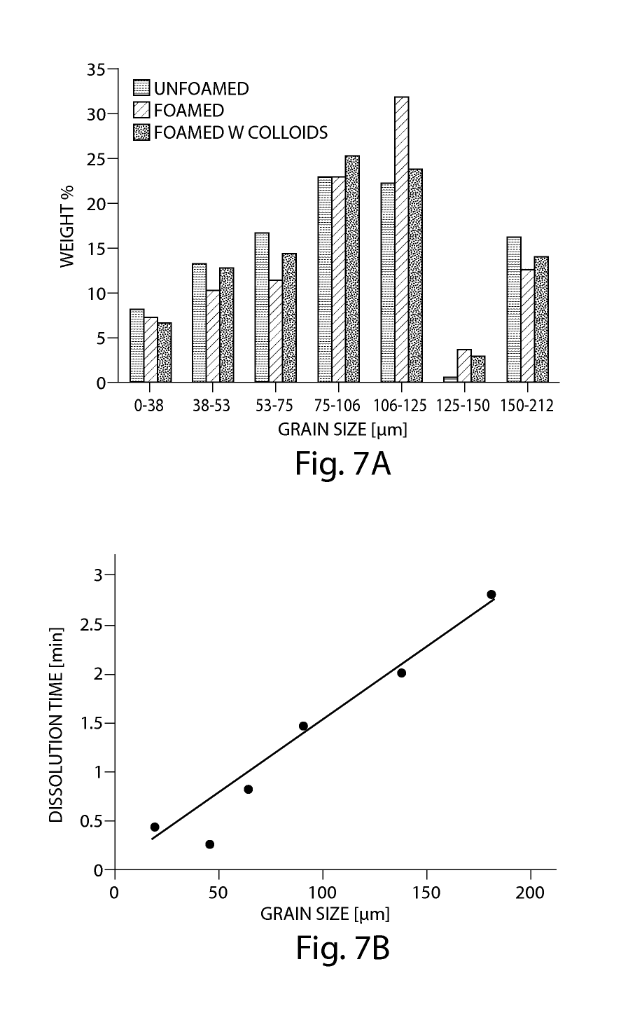

The final step before dissolution was to mill the samples. Milling breaks the bubbles open in the foamed samples so that the interior surface area is accessible to a dissolving liquid. It was found, however, that extended milling may destroy the interior structure of the foam. Because the goal of this example was to investigate the effect of the interior structure on dissolution, and not to create very small particles through milling alone, a gentle cryo-milling technique that broke the samples into large, 10 micrometer to 100 micrometer “chunks” that retained the porous structure of the foam was used in this example. The same milling protocol was used for all of the formulations, including solid solutions and foamed solid solutions. The resulting grain size distribution of all samples was found to be similar (FIG. 7A). Thus, the grain size distribution appeared to be controlled by milling conditions. This allowed a direct comparison of the dissolution rates of the different formulations.

FIG. 1A shows a schematic diagram of the foam templating process, including polymer matrix, and dissolved drug molecules. At high pressure, the polymer absorbed CO₂, which precipitated out and nucleated bubbles when the pressure was released. The bubbles grow and pack densely, increasing the surface area.

To establish a link between surface-to-volume ratio and dissolution rate, dissolution times of milled, sieved solid solutions were determined as a function of grain size. The dissolution time τ(tau) may be defined as the time to dissolve 63% of the active; this definition allows these results to be compared to predictions from the Nernst-Brunner equation, which describes simple diffusion and is often used to model the dissolution rate of pharmaceutical actives:

$$\frac{d C}{d t} = \frac{DA}{Vl} ( {C}_{s} - C ) , \qquad ( 1 )$$

where C is the instantaneous concentration, Cₛ is the saturation concentration (solubility), D is the diffusivity, l is the thickness of the diffusion layer, V is the volume of the medium, and A is the total surface area of dissolving particles. The dissolution time corresponded to the characteristic time τ=Vl/DA in the exponential C˜1−exp(t/τ), which is the solution to Eq. 1. As shown in FIG. 7B, the measured dissolution time τ scales linearly with the grain size, in agreement with Nernst-Brunner.

FIG. 7 illustrates the grain size distribution for foams with clotrimazole, as determined by sieving. FIG. 7A shows the grain size distribution for unfoamed solid solutions (left), foamed solid solutions (center), and foamed solid solutions with colloidal particles (right) after milling. (See a PCT application filed on even date herewith, entitled “Foams Including Microcellular Foams Containing Colloidal Particulates,” by Ladavac, et al., incorporated herein by reference, for additional details.) FIG. 7B shows the characteristic dissolution time τ as a function of grain size for unfoamed solid solutions.

In some experiments, it was found that for a given solid solution, the foam morphology was determined by operating pressure, temperature, and pressure release rate. The operating parameters controlled the amount of gas delivered to the polymer, the bubble nucleation rate, and time allowed for bubbles to grow before the polymer vitrifies. Increasing the pressure increased the CO₂ density, yielding more fluid dissolved in the polymer matrix and, in general, a higher final bubble volume fraction and smaller length scales. Decreasing the temperature increased the CO₂ density, leading to more gas dissolved in the polymer, but when the pressure is released there is less time for the bubbles to flow before the structure vitrifies. Decreasing the pressure release time induced a larger thermodynamic instability and a higher oversaturation of CO₂ in the polymer. This lead to a higher nucleation rate, higher volume fraction, and smaller length scales.

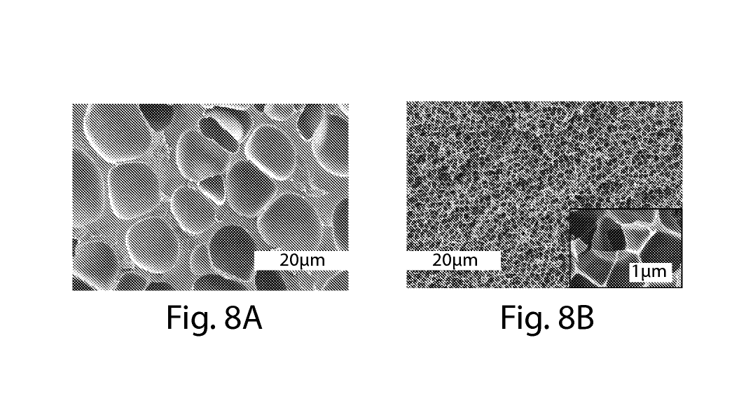

If these parameters are not optimized, the resulting foams may exhibit, in some instances, large bubbles, small bubble volume fractions, thick films, and/or large Plateau borders. For example, foams produced at 100 atm CO₂ pressure with a pressure release time of 3 s exhibited films a few micrometers thick and Plateau borders of approximately 10 micrometers, as shown in FIG. 8A. FIG. 8A is an SEM image of an unoptimized foam made at 100 atm, 50° C., 3 s pressure release time. By increasing the pressure to the maximum allowed by the apparatus used in this example, 400 atm, reducing the pressure release time to ˜200 ms, and optimizing the temperature, foams with micrometer-scale bubbles and volume fractions of 90% were produced. The film thickness was on the order of tens of nanometers, more than an order of magnitude smaller than the bubbles, and the Plateau borders were on the order of hundreds of nanometers, as shown in FIG. 8B, showing an SEM image of an optimized foam made at 400 atm, 40° C., ˜200 ms pressure release time. The inset shows a magnified view of optimized Plateau borders and films.

All materials were used in these experiments as received, including PVPVA, poly(1-vinylpyrrolidone-co-vinyl acetate) 6:4, (Kollidon VA 64, BASF, CAS 25086-89-9); CO₂, carbon dioxide (Coleman Grade-Min. Purity 99.99% Liquid Phase); clotrimazole (Selectchemie, Lot No. 20051116); itaconazole (Selectchemie, Batch No. IT0070709); fenofibrate (Aldrich, Batch No. 017K1401); carbamazepine (Pfannenschmitt, Batch No. 07092639); cholesterol (Alfa Aesar, 96% pure, CAS 57-88-5).

Preparing solid solutions. Two methods to prepare solid solutions of active in polymer. When small amounts were needed, a co-solvent method was used, in which active and polymer were dissolved in a common solvent, either acetone or ethanol, which was then removed by evaporation. To accelerate drying, the solution was spread on a sheet, dried overnight at 50° C., milled, and dried again. The powders were then heated to a molten state at 120° C. and pressed (Carver 24-ton hydraulic press) to make the final bulk pellets. Because small amounts of residual solvent may substantially reduce T_{g}, the solid solutions were analyzed with a Thermogravimetric Analyzer (TGA, TA Instruments Q50001R) and a Differential Scanning calorimeter (DSC, TA Instruments Q200). Fully dried samples showed no significant loss of weight through heating, indicating no residual solvent, and a single glass transition temperature, a signature of true solid solution.

For larger amounts of sample (˜10 g), hot melt extrusion was used. Polymer and drug were directly mixed in a small-scale twin-screw extruder (Micro Compounder, DACA Instruments). To ensure full dissolution of the drug in polymer, the extrusion was performed above the melting point of the drug. For example, an operating temperature of 160° C. for clotrimazole was used. High performance liquid chromatography (Agilent 1100 HPLC) showed that the drug did not degrade during heating.

Foaming. To foam the solid solutions, a custom-built apparatus was prepared, including a CO₂ cylinder, pump, and chamber. Gas was drawn from the cylinder to a high pressure syringe pump (model 260D, Teledyne Isco, Lincoln Nebr.) connected to a 100 ml hand-tight steel chamber (made by Pressure Products Industries Inc., Warminster, Pa., purchased from Supercritical Fluid Technologies Inc., Newark, Del.). The pressure was set by the pump, and the temperature was set by a heating sheet wrapped around the chamber. The heating sheet was powered through a PID controller (Omega Engineering, CSI32K iSeries Benchtop controller) that maintains the working temperature with a feedback loop through a thermocouple (Omega Engineering, KHSS-18G-RSC) mounted in the chamber. The pressure release times were made as small as possible by reducing the amount of dead volume in the chamber and quickly venting the CO₂ through a pneumatically activated 3-way valve (Swagelok, SS-H83XPF2-53S). This experimental apparatus handled up to 500 atm pressure, 200° C. temperature, and pressure release times as short as 100 ms. In a typical experiment, 1 gram of solid solution was added to the chamber, allowed to soak for 4 hours at 40° C. and 400 atm pressure, and then pressure is released within 200 ms.

Choice of polymer. The polymer to be used for the solid solution was selected in this example to absorb enough CO₂ at reasonable pressures to make a foam with high volume fraction, and its T_{g} must allow a working temperature that is low enough for this apparatus, but above 31.1° C., to ensure the CO₂ was supercritical. For this particular example application, oral drug delivery, the polymer also needed to be water soluble and approved for ingestion.

In these experiments, PVPVA, a random copolymer of PVP (poly(vinylpyrrolidone)) and PVAc (poly(vinyl acetate)), was selected. PVAc provided high affinity for CO₂ (absorbs more than 20% w/w at 25° C.), while PVP made the whole copolymer more water soluble and is itself a good solvent for many drugs. The glass transition temperature of the pure copolymer was found to be 108° C., above the critical point of CO₂, but well within the working temperature of this experimental apparatus. Adding actives to the polymer typically reduced the T_{g} of the polymer; this was compensated for by adjusting the temperature of the foaming process.

Imaging. A Zeiss Ultra/Supra scanning electron microscope was used to image the foam samples. To expose the structure for imaging without damaging it, the foam was frozen in liquid nitrogen to make the polymer brittle and carefully fractured with a sharp blade. The polymer was nonconducting, so to reduce charging under the electron beam, a thin layer of platinum/palladium was sputtered there.

Milling and characterizing. The samples were loaded in a 50 ml stainless steel jar with one 25 mm stainless steel ball and milled for 2 minutes at 10 Hz while the jar flushed with liquid nitrogen (CryoMill, Retsch Corp.) The surface area of the milled samples was measured by nitrogen adsorption through the BET method (Beckman Coulter Surface Area Analyzer SA3100). To measure the size distribution of milled powders, the powders were first separated by grain size using a Cole Parmer Sieve Shaker, vibrating at 60 Hz, 1 s tapping, with a stack of stainless steel sieves (ASTM E-11 standard) decreasing in mesh size from top to bottom. After sieving for 20 minutes the final contents of each sieve were weighed to determine the grain size distribution.

Dissolution tests. To measure dissolution rates, a custom-built apparatus was used that included a dissolution chamber, a peristaltic pump, and a UV-VIS spectrophotometer. The dissolution chamber (Millipore Solvent-Resistant Stirred Cell 76 mm) was connected through a peristaltic pump to a quartz flow cell (Starna Cells) mounted in the UV-VIS spectrophotometer (Perkin Elmer Lambda 40). The bottom of the chamber was fitted with a filter membrane (Sterlitech PTFE laminated membrane, either 0.2 or 0.45 micrometer pore size) that prevented any undissolved particles from reaching the spectrophotometer. A magnetic stiffing bar was mounted in the dissolution chamber and actuated by a magnetic stir-plate below the chamber. The solution flowed through continuously, with the spectrometer measuring the absorbance of the dissolved drug. To relate the absorbance to concentration, samples of known concentration were measured in a good solvent such as ethanol.

After reaching the spectrometer cell, the solution was recirculated back to the chamber, keeping the total volume constant. The total delay time between the addition of the sample to the chamber and the appearance of a steady signal on the spectrophotometer was approximately 30 seconds. This set the time resolution of the measurement.

Samples were applied as powder directly into the dissolution chamber. To help the powder sink and prevent clumping, the powder was mixed with a spacer, an inert material that does not interact chemically with either the active or the polymer. Either microcrystalline cellulose (20 micrometer powder, Aldrich) or fumed silica (sintered aggregates 200 nm large, composed of 10 nm particles, CAB-O-SIL M5, Cabot Corp.) was used. The choice of spacer did not affect the measured dissolution rate.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A pharmaceutically active article, comprising:
a foam comprising a pharmaceutically acceptable polymeric carrier and a pharmaceutically active agent, the foam having an average cell size of less than about 5 micrometers, wherein the foam (a) has a specific surface area of at least about 0.4 m²/g, and/or (b) has a foam density of less than about 1 g/cm³.

2. The pharmaceutically active article of claim 1, wherein the pharmaceutically active agent is present at least about 30 wt %.

3-10. (canceled)

11. The pharmaceutically active article of claim 1, wherein the foam has a specific surface area of at least about 0.5 m²/g.

12-17. (canceled)

18. The pharmaceutically active article of claim 1, wherein the foam has an average cell size of less than about 4 micrometers.

19-22. (canceled)

23. The pharmaceutically active article of claim 1, wherein the pharmaceutically acceptable polymeric carrier exhibits a glass transition temperature of at least about 90° C.

24-25. (canceled)

26. The pharmaceutically active article of claim 1, wherein the pharmaceutically acceptable polymeric carrier comprises poly(vinylpyrrolidone).

27. The pharmaceutically active article of claim 1, wherein the pharmaceutically acceptable polymeric carrier comprises poly(vinyl acetate).

28-30. (canceled)

31. The pharmaceutically active article of claim 1, wherein the foam has a cellular number density of at least about 10⁸ cm⁻³.

32-33. (canceled)

34. The pharmaceutically active article of claim 1, wherein the foam is a blown foam.

35. (canceled)

36. The pharmaceutically active article of claim 1, wherein the foam has a void fraction of at least about 50 vol %.

37-41. (canceled)

42. A pharmaceutically active article, comprising:
a plurality of particles, the particles comprising a pharmaceutically acceptable polymeric carrier and a pharmaceutically active agent and having an average characteristic dimension of no more than about 5 micrometers and a specific surface area of at least about 6 m²/g, wherein (a) at least about 20% of the particles have at least two concave surface regions, and/or (b) in at least about 20% of the particles, at least about 50% of the external surface area of the particles is present within a concave surface region.

43. The pharmaceutically active article of claim 42, wherein at least about 20% of the particles have at least two concave surface regions.

44. The pharmaceutically active article of claim 42, wherein, in at least about 20% of the particles, at least about 50% of the external surface area of the particles is present within a concave surface region.

45-48. (canceled)

49. The pharmaceutically active article of claim 42, wherein the specific surface area is at least about 7 m²/g.

50-51. (canceled)

52. The pharmaceutically active article of claim 42, wherein the pharmaceutically active agent is present in the plurality of particles in an amount of at least about 5% based on the weight of the plurality of particles.

53. (canceled)

54. The pharmaceutically active article of claim 42, wherein the plurality of particles exhibits a glass transition temperature of between about 90° C. and about 110° C.

55-57. (canceled)

58. A method of forming a pharmaceutically active article, comprising:
mixing a pharmaceutically acceptable polymeric carrier and a pharmaceutically active agent with a foaming agent to form a precursor of a foam, wherein the foaming agent is present in an amount of at least about 5% by weight based on the weight of the mixture, and the pharmaceutically active agent is present in an amount of at least about 5% based on the weight of the mixture; and
subjecting the precursor to a pressure drop whereby the foaming agent expands and forms the pharmaceutically active article as a foam of the precursor, wherein the foam is microcellular.

59. (canceled)

60. The method of claim 58, wherein the foaming agent comprises CO₂.

61. The method of claim 58, wherein the foaming agent is mixed with the pharmaceutically acceptable polymeric carrier under conditions such that the foaming agent is supercritical.

62. The method of claim 58, comprising mixing the pharmaceutically acceptable polymeric carrier and the foaming agent at a temperature of at least about 30° C.

63-64. (canceled)

65. The method of claim 58, comprising mixing the pharmaceutically acceptable polymeric carrier and the foaming agent at a pressure of at least about 50 atm.

66-73. (canceled)

74. The method of claim 58, wherein the pressure drop is applied for a time of less than about 1 s.

75-77. (canceled)

78. The method of claim 58, wherein the precursor is formed by mixing the pharmaceutically acceptable polymeric carrier and the pharmaceutically active agent with a cosolvent, and removing the cosolvent.

79. The method of claim 78, wherein the precursor formed after removing the cosolvent is solid at room temperature.

80. (canceled)

81. The method of claim 79, wherein the solid precursor is ground to form particles.

82-84. (canceled)

85. The method of claim 58, wherein the precursor is exposed to a pressure of at least about 5,000 lb/in².

86. (canceled)

87. The method of claim 58, wherein the precursor is exposed to a temperature of at least about 80° C.

88-106. (canceled)
